(19) United States
(12) Patent Application Publication
(10) Pub. No.: US 2019/0108315 A1
(43) Pub. Date: Apr. 11, 2019

(54) SYSTEM AND METHOD FOR IMPROVING HEALTHCARE THERAPY RECOMMENDATIONS

(71) Applicant: Healthcare Sales Enablement, Inc., Coral Gables, FL (US)

(72) Inventors: David John Thornton, Coral Gables, FL (US); Anatoly Valentin Geyfman, Portland, OR (US); Daniel Joseph Imfeld, Lihue, HI (US)

(73) Assignee: Healthcare Sales Enablement, Inc., Coral Gables, FL (US)

(21) Appl. No.: 15/730,245

(22) Filed: Oct. 11, 2017

(51) Int. Cl. G06F 19/00; G06Q 10/02; G06Q 50/24

(52) U.S. Cl. CPC G06F 19/322; G06Q 50/24; G06Q 10/02

(57) ABSTRACT

A method of improving healthcare therapy recommendations, the method including establishing a healthcare dataset including at least one set of predetermined medical criteria associated with the at least one identifiable healthcare therapy, identifying by a processor a plurality of healthcare providers who treated at least one patient meeting the predetermined medical criteria and at least one patient meeting the set of predetermined medical criteria, and storing at least one of the plurality of healthcare providers and the at least one patient meeting the set of predetermined medical criteria on a storage medium.

### CROSS REFERENCE

This application is a non-provisional application of, and claims priority to, provisional application No. 62/406,713, filed Oct. 11, 2016, and titled “System and Method for Improving Healthcare Therapy Recommendations,” the entirety of which is hereby incorporated by reference.

### FIELD OF THE INVENTION

The present invention relates generally to healthcare, and, more particularly, relates to a system and method of identifying one or more healthcare providers and patients meeting a set of predetermined medical criteria.

### BACKGROUND OF THE INVENTION

Although there are many systems and methods used to match patients with the appropriate healthcare providers, little attention has been paid to effective solutions which readily identify healthcare providers who treat select patients that may benefit from a certain healthcare therapy, but are unaware of the existence of the particular healthcare therapy. Said another way, for a given disease state, there is often a large gap between the number of patients who may benefit from one or more therapies developed for that disease state and the number of patients who are actually treated with those therapies. Depending on the disease state, the failure to render select therapies to patients in need often leaves the patients in significantly worse condition than would otherwise occur if the therapies were provided to the patient. Obviously, the lack of therapy is not only detrimental to the patient, but also imposes an enormous and unnecessary cost burden on the overall healthcare system.

The failure to match the beneficial therapies to the appropriate healthcare providers and patients exists in part because the parties responsible for introducing the new therapies lack the information needed to sufficiently target and educate the qualified providers and appropriate patients. Traditionally, the producer of a therapy targets a particular class of healthcare providers for educational outreach, with the targeting criteria based on provider-related information, such as medical specialties, prescription offerings, and/or the types of medical procedures performed by the healthcare provider. Unfortunately, the manner in which healthcare providers are targeted often includes narrow criteria which fails to identify each healthcare provider who customarily treats the type of patients who may benefit from the select therapy. As a result, the providers treating a large number of patients who may benefit from a given therapy are never actually educated about the existence of such therapy. Obviously, the failure to educate or bring awareness to the particular therapy often leaves patients without the therapy or the therapy may be offered to patients with less frequency than is appropriate.

Therefore, a need exists to overcome the problems with the prior art as discussed above.

### SUMMARY OF THE INVENTION

Embodiments of the invention provide a method of improving healthcare therapy recommendations that includes establishing a healthcare dataset including at least one set of predetermined medical criteria associated with at least one identifiable healthcare therapy. The method of the embodiments further includes identifying, by a processor, a plurality of therapy healthcare providers who treated at least one patient meeting the predetermined set of medical criteria and an identifier of at least one patient meeting the set of predetermined medical criteria. The method of the embodiments further includes storing at least one of the plurality of therapy healthcare providers and the identifier of the at least one patient meeting the set of predetermined medical criteria on a storage medium.

A further object of the embodiments can include identifying the plurality of therapy healthcare providers comprises comparing therapy criteria with a plurality of patient records in the healthcare dataset.

Another further object of the embodiments can include comparing a primary therapy criteria with at least one primary patient medical parameter, and comparing a secondary therapy criteria with at least one secondary patient medical parameter.

Another further object of the embodiments can include, subsequent to storing at least one of the plurality of therapy healthcare providers and the identifier of the at least one patient, alerting a general healthcare provider associated with the at least one patient meeting the set of predetermined medical criteria, and indicating a matching therapy and therapy provider of the therapy.

Another object of the embodiments can include receiving the healthcare dataset from a general healthcare provider, and receiving the medical criteria from a therapy developer.

Another object of the embodiments can include scoring a comparison of patent medical parameters with the set of medical criteria, and identifying patients whose patient medical parameters meet or exceed a minimum score.

Some embodiments of the invention provide a method of facilitating therapy recommendations to patients including comparing, for each of a plurality of patients, a healthcare dataset associated with the patient with at least one medical criteria associated with a respective one of at least one therapy. The method of the embodiments can further include identifying patients among the plurality of patients whose healthcare dataset sufficiently matches at least one medical criteria. The method of the embodiments can further include indicating to a general healthcare provider associated with each of the identified patients whose healthcare dataset matches at least one medical criteria, including indicating an identity of a therapy healthcare provider associated with the at least one medical criteria.

Some embodiments of the invention provide a system of improving healthcare therapy recommendations that includes at least one database having stored therein a healthcare dataset including at least one set of predetermined medical criteria. The system of the embodiments further includes a processing device that is communicatively coupled to the at least one database, and a user-input interface that is communicatively coupled to the processing device. The user-input interface is configured to receive input instructions from the user to identify a plurality of healthcare providers who treated at least one patient meeting the set of predetermined medical criteria and identify at least one patient meeting the set of predetermined medical criteria. The system of the embodiments further includes a memory configured to store at least one of the plurality of healthcare providers and the at least one patient meeting the set of predetermined medical criteria on a storage medium.

### BRIEF DESCRIPTION OF THE DRAWINGS

The accompanying figures, where like reference numerals refer to identical or functionally similar elements throughout the separate views and which together with the detailed description below are incorporated in and form part of the specification, serve to further illustrate various embodiments and explain various principles and advantages all in accordance with the present invention.

FIG. 1 shows a flow chart diagram of a method for improving healthcare therapy recommendations, in accordance with some embodiments;

FIG. 2 shows a schematic block diagram of a data processing system that matches patients with care providers, in accordance with some embodiments;

FIG. 3 shows a system diagram of an exemplary therapy matching network, in accordance with some embodiments; and

FIG. 4 is a flow chart diagram of a method of matching patients with therapies, in accordance with some embodiments.

### DETAILED DESCRIPTION

Although the invention is illustrated and described herein as embodied in a system and method for improving healthcare therapy recommendations, it is, nevertheless, not intended to be limited to the details shown because various modifications and structural changes may be made therein without departing from the spirit of the invention and within the scope and range of equivalents of the claims. Additionally, well-known elements of exemplary embodiments of the invention will not be described in detail or will be omitted so as not to obscure the relevant details of the invention.

Other features that are considered as characteristic for the invention are set forth in the appended claims. As required, detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one of ordinary skill in the art to variously employ the present invention in virtually any appropriately detailed structure. Further, the terms and phrases used herein are not intended to be limiting; but rather, to provide an understandable description of the invention. While the specification concludes with claims defining the features of the invention that are regarded as novel, it is believed that the invention will be better understood from a consideration of the following description in conjunction with the drawing figures, in which like reference numerals are carried forward. The figures of the drawings are not drawn to scale.

Before the present invention is disclosed and described, it is to be understood that the terminology used herein is for the purpose of describing particular embodiments only and is not intended to be limiting. The terms “a” or “an,” as used herein, are defined as one or more than one. The term “plurality,” as used herein, is defined as two or more than two. The term “another,” as used herein, is defined as at least a second or more. The terms “including” and/or “having,” as used herein, are defined as comprising (i.e., open language). The term “coupled,” as used herein, is defined as connected, although not necessarily directly, and not necessarily mechanically. The term “providing” is defined herein in its broadest sense, e.g., bringing/coming into physical existence, making available, and/or supplying to someone or something, in whole or in multiple parts at once or over a period of time.

As used herein, the terms “about” or “approximately” apply to all numeric values, whether or not explicitly indicated. These terms generally refer to a range of numbers that one of skill in the art would consider equivalent to the recited values having the same function or result). In many instances these terms may include numbers that are rounded to the nearest significant figure. The terms “program,” “software application,” and the like as used herein, are defined as a sequence of instructions designed for execution on a computer system. A “program,” “computer program,” or “software application” may include a subroutine, a function, a procedure, an object method, an object implementation, an executable application, an applet, a servlet, a source code, an object code, a shared library/dynamic load library and/or other sequence of instructions designed for execution on a computer system.

The accompanying figures, where like reference numerals refer to identical or functionally similar elements throughout the separate views and which together with the detailed description below are incorporated in and form part of the specification, serve to further illustrate various embodiments and explain various principles and advantages all in accordance with the present invention.

While the specification concludes with claims defining the features of the invention that are regarded as novel, it is believed that the invention will be better understood from a consideration of the following description in conjunction with the drawing figures, in which like reference numerals are carried forward. It is to be understood that the disclosed embodiments are merely exemplary of the invention, which can be embodied in various forms.

The present invention provides a novel and efficient computer-facilitated system and method for improving healthcare therapy recommendations. More specifically, in one embodiment, the system and method herein may include identifying one or more healthcare providers who have treated, or are likely to treat, at least one patient meeting a set of predetermined medical criteria. Embodiments of the invention also provide the system and method including identifying at least one patient meeting the set of predetermined medical criteria. The information associated with the identified healthcare providers and the patients may be stored on a storage medium. Advantageously, such information may be accessed and used by therapy developers to target the appropriate healthcare providers and patients. More specifically, those responsible for educating others about a given therapy may reach an increasing number of the providers that are qualified to render the particular type of therapy. For example, a pharmaceutical cancer drug representative may be provided with the identity information for an oncologist ho has treated one or more patients having a specific type of cancer and who may benefit from the pharmaceutical cancer drug offered by the representative. The present system and method not only increases revenue for the therapy developers and the healthcare providers, but also increases the likelihood of treating a number of patients that would not otherwise have been offered the therapy, without being identified through the system and method described herein.

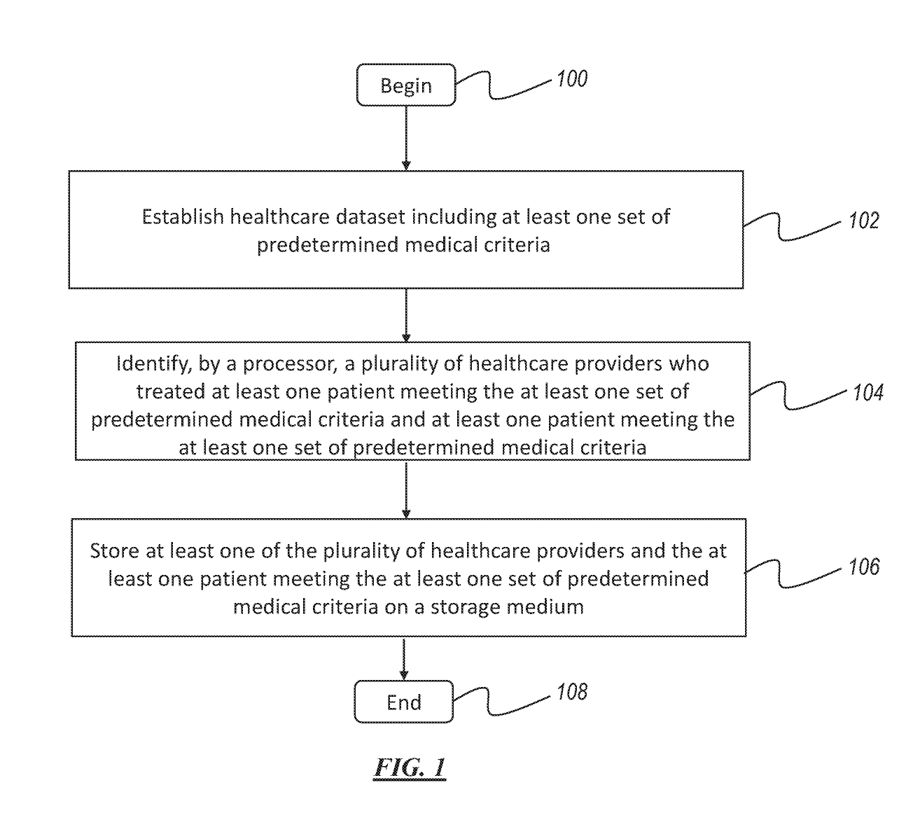

Referring now to FIG. 1, one embodiment of the present invention is shown as a process flow diagram. More specifically, FIG. 1 provides a method of improving healthcare therapy recommendations. FIG. 1 shows several advantageous features of the present invention, but, as will be described below, the invention can be provided in several shapes, sizes, combinations of features and components, and varying numbers and functions of the components. The steps delineated in the exemplary process flow diagram of FIG. 1 are merely exemplary of the preferred order of improving healthcare therapy recommendations, and said steps may be carried out in another order, with or without additional steps included therein. Also, two or more blocks shown in succession may be executed concurrently or with partial concurrence in some embodiments. Certain steps may also be omitted in FIG. 1 for the sake of brevity. In some embodiments, some or all of the process steps included in FIG. 1 can be combined into a single process.

In one embodiment, the method begins at step 100 and moves directly to step 102, where a healthcare dataset may be established including at least one set of predetermined medical criteria. In one embodiment, the healthcare dataset may be established by a healthcare provider, a person or entity responsible for developing healthcare therapies, or any other entity familiar with healthcare treatment regimens. The healthcare dataset and the set of predetermined medical criteria correspond to one or more therapies that may prove to be beneficial for a patient having a particular set of symptoms. Said another way, the t of medical criteria is intended to identify a set of patients who may benefit from a particular therapy. In one exemplary embodiment, the set of predetermined medical criteria includes diagnoses that a patient has received. In other embodiments, the set of predetermined medical criteria may include prescription drugs prescribed to the patient, procedures that have been performed on the patient, and/or temporal relationships between the diagnoses, prescriptions, and procedures. The term “therapy” may be used synonymously with the term “treatment” and is defined herein in its broadest possible sense as the treatment of one or more physical, mental, social disorders, or diseases. For example, the term therapy may include, but is not limited to, implantable medical devices, prescription drugs, surgical techniques, and the like.

In one embodiment, the healthcare datasets may include information relating to patient identity. More specifically, the patient identity information may include, but is not limited to, age, gender, geographic location, diagnoses, and healthcare providers visited by the patient. The term “healthcare provider” may include organizational healthcare providers, e.g., hospitals, individual healthcare providers, e.g., physicians, or any other person or entity providing healthcare services, medical equipment, prescription drugs, or the like, to patients.

In one embodiment, the method may proceed to step 104 including identifying, by a processor, one or more healthcare providers who treated at least one patient meeting the set of predetermined medical criteria and at least one patient meeting the set of predetermined medical criteria. The processor is a hardware computing device which runs computer instruction code that is designed in accordance with the teachings herein. In one embodiment, the healthcare providers and/or the patients may be identified using information from at least one database. In one embodiment, the database may be a healthcare claims database, such as a healthcare claims database of a healthcare insurance provider. In other embodiments, the database may be provided from one or more sources including, but not limited to, private pay sources, government agencies, and the like.

In one embodiment, the present method may be implemented through a software application running on an electronic device, such as a smart phone, tablet, desktop computer, laptop, or the like. The software may operate in conjunction with a server, a processor, a user-input interface, and a storage medium, as described in further detail below. In one embodiment, a user, such as a therapy developer, may input via a user-input interface, e.g., a touchscreen, instructions for a processor to search through the database to identify one or more healthcare providers who treated at least one patient meeting the set of predetermined medical criteria and at least one patient meeting the set of predetermined medical criteria.

In one embodiment, the method may proceed to step 106 including storing at least one healthcare provider and at least one patient meeting the set of predetermined medical criteria on a storage medium, which is a tangible, non-transitory storage medium. The storage medium may be used in conjunction with the memory controller/cache explained in further detail below. In one embodiment, as mentioned above, the identity information may be accessed by those responsible for developing and/or educating healthcare providers about specific therapies. Advantageously, those responsible for educating healthcare providers about the therapies are provided a target market of healthcare providers best suited to provide the therapy to patients who may benefit from the therapy or a given set of therapies. Said another way, the present system and method improves therapy recommendations by allowing therapy developers to target and educate the appropriate healthcare: providers about therapies that are likely to be beneficial to the type of patient treated by the healthcare providers. The method ends at step 108.

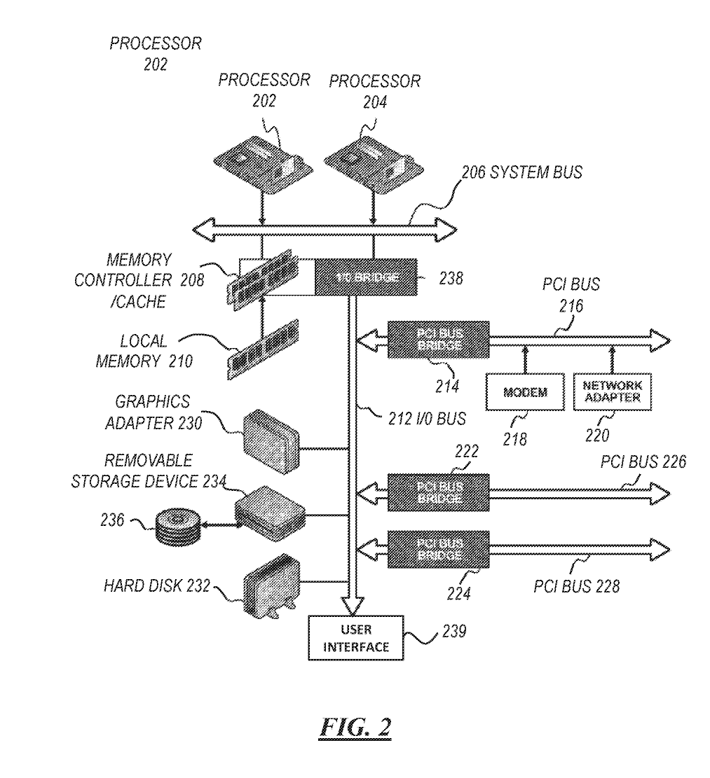

Referring now primarily to FIG. 2, with brief reference to FIG. 1, there is depicted an exemplary data processing system 200 in a block diagram, which may be implemented as a server configured to carry out the method described herein. The data processing system 200 may be a symmetric multiprocessor (SMP) system including a plurality of processors 202 and 204 connected to system bus 206. Alternatively, a single processor system may be employed. Also, connected to system bus 206 is memory controller/cache 208, which provides an interface to local memory 210. An I/O bus bridge 238 is connected to system bus 206 and provides an interface to I/O bus 212. The memory controller/cache 208 and I/O bus bridge 238 may be integrated as depicted. The processor 202 or 204 in conjunction with memory controller 208 controls what data is stored in memory 210. The processor 202 and/or 204 and memory controller 208 can serve as a data counter for counting the rate of data flow to the memory 210 or from the memory 210 and can also count the total volume of data accessed to or from the memory 210. The processor 202 or 204 can also work in conjunction with any other memory device or storage location.

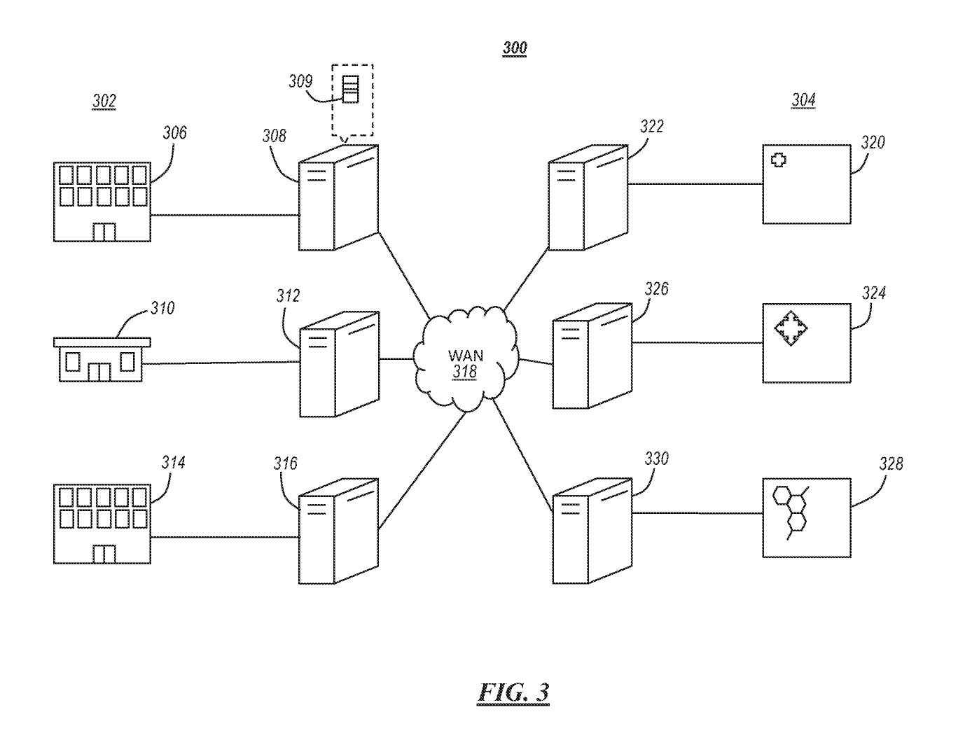

A peripheral component interconnect (PCI) bus bridge 214 is connected to I/O bus 212 and provides an interface to PCI local bus 216. A number of modems 218, or wireless cards, may be connected to PCI bus 216. Typical PCI bus implementations will support four PCI expansion slots or add-in connectors. PCI includes, but is not necessarily limited to, PCI-X and PCI Express components. Communications links to the network of computers in FIGS. 2 and 3 may be provided through the modern 218 and network adapter 220 connected to PCI local bus 216 through add-in boards.

Additional PCI bus bridges 222 and 224 provide interfaces for additional PCI buses 226 and 228, from which additional moderns or network adapters may be supported. In this manner, the data processing system 200 allows connections to a multiple network of computers. A graphics adapter 230 and hard disk 232 may also be connected to 170 bus 212 as depicted, either directly or indirectly. A user-input interface 239 is operably coupled to the I/O bus 212, and includes 110 devices such as, for example, a graphical display interface, a keyboard interface, a universal serial bus, a pointing device interface. In general, the user-input interface 239 is communicatively coupled to the processing device or devices 202, 204, and is configured to receive input instructions from a user to identify a plurality of healthcare providers who treated at least one patient meeting the set of predetermined medical criteria and identify at least one patient meeting the set of predetermined medical criteria.

Those of ordinary skill in the art will appreciate that the hardware depicted in FIG. 2 may vary. For example, other peripheral devices, such as optical disk drives and the like, also may be used in addition to or in place of the hardware depicted. The depicted example is not meant to imply architectural limitations with respect to the present invention.

FIG. 3 shows a system diagram of an exemplary therapy matching network 300, in accordance with some embodiments. Several healthcare providers 302 can include, for example, a hospital 306, a medical office 310, and a healthcare center 314. Each of the healthcare providers actively provide healthcare services to patients, and collect data regarding their conditions, symptoms, diagnoses, and demographic information, which can be organized into healthcare datasets and medical criteria, as referenced in FIG. 1. Each healthcare provider 302 exposes the healthcare datasets and medical criteria on a respective networked server 308, 312, 316, which contain records 309 containing information for various patients. The information can be protected so as to not expose a patient's identity. Furthermore, the records 309 may be exposed with consent of the patients.

Similarly, a plurality of healthcare providers 304 can include, for example, a research hospital 320, a research institute 324, and a pharmacological research organization 328. Each healthcare provider 320, 324, 328 applies and/or develops one or more medical therapies to treat specific medical conditions, and can utilize a respective computing system 322, 326, 330 to access a wide area network 318, to which each of the healthcare servers 308, 312, 316 are connected. Each of the general healthcare providers 306, 310, 314 can receive the therapy healthcare providers 320, 324, 328 via their respective server 308, 312, 316, therapy information or therapy criteria of patients they have treated using those therapies. The information allows the healthcare providers, or third parties, to match patients with therapy healthcare providers that have provided therapies for patients with similar medical parameters. Thus each therapy healthcare providers' computing systems 322, 326, 330 includes and can transmit a set of criteria that represents the primary and secondary medical parameters that qualify for the therapy, which can represent medical parameters of patients that have responded well to the therapies. The primary medical parameter indicate conditions of the patient's body, such as, for example, blood lipid panel measurements, blood pressure values, and so on, whereas secondary medical parameters can include information such as gender, weight, age, prescriptions, and other such information.

The general healthcare providers computing systems 308, 312, 316 can be constructed in accordance with that shown in FIG. 2, and records stored by the healthcare providers 306, 310, 314 to compare patient information in records (e.g. 309), including primary and secondary medical parameters, with therapy criteria to identify potential candidates for the therapy or therapies provided by the therapy healthcare providers 320, 324, 328. For every patient record that matches a therapy criteria, the computing systems 308, 312, 316 can alert the respective healthcare provider 306, 310, 314 indicating a patient identifier as a possible therapy candidate, and identify the therapy provider or providers that have treated patients with similar medical parameters. An identified patient can then discuss the therapy with a healthcare provider to determine whether it is suitable and/or desirable to undergo or undertake the therapy.

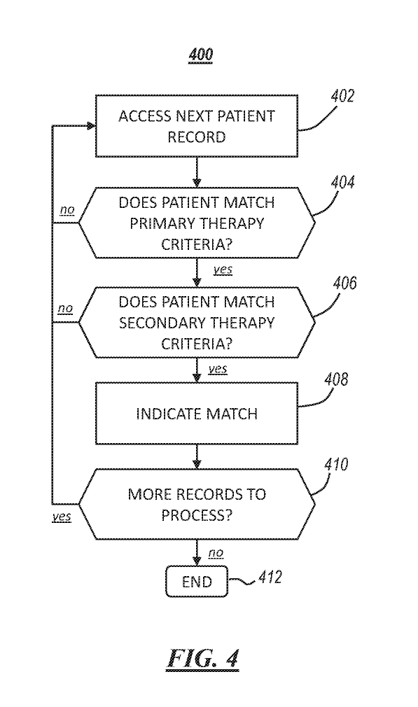

FIG. 4 shows a flow chart diagram of a method 400 of matching patients with therapies, in accordance with some embodiments. To start, the method 400 commences accessing a patient record, or a next patient record in step 402. The patient records are made available by healthcare providers and represent patients under their care, and can include primary and secondary medical parameters. In step 404 the method 400 can compare the patient's primary medical parameters with primary therapy criteria. Specifically, whether the patient's primary medical parameters fall within a range, or above or below a given threshold of the primary therapy criteria. If the patient's primary medical parameters indicate a sufficient match to the primary therapy criteria, then the method commences to step 406 where a similar comparison is made between the patient's secondary medical parameters and the secondary therapy criteria. Steps 404, 406 are shown as two separate steps here, but can be combined into one process. Furthermore, for each parameter being compared, a scoring scale can be used to indicate how much of a match or overlap there is between the patient's medical parameter and the corresponding therapy parameter. A total score can be derived by weighing the scores for each parameter, and the weighting scalars can be provided by the therapy developer. In addition, the weighting scalars can be dependent on the patient's medical parameters. For example, a patient's age or weight can adjust weighting scalars for each parameter. If in steps 404, 406, there is an insufficient match, then the method 400 returns to step 402 to commence processing the next record. If a sufficient match is found between the patient's medical parameters and the therapy criteria, then the method proceeds to step 408 to indicate that the patient is a potential match for receiving the therapy. IN step 410 the method 400 checks if there are more patient records to process and if so, returns to step 402, otherwise the method 400 ends 412.

The processes explained in detail above can be embodied in a computer program. Computer programs (also called computer control logic) are stored in memory such as main memory 210, removable storage drive 234, removable media 236, hard disk 232, and signals. Such computer programs, when executed, enable the computer system to perform the features of the present invention as discussed herein. In particular, the computer programs, when executed, cause the processor 202 and/or 204 to perform the features of the system and method of improving therapy recommendations 100.

In this document, the terms “computer program medium,” “computer usable medium,” and “computer readable medium” are used to generally refer to media such as local memory 210, removable storage drive 234, removable media 236, hard disk 232, and signals. These computer program products are means for providing software to the computer system. The computer readable medium allows the computer system to read data, instructions, messages or message packets, and other computer readable information from the computer readable medium. The computer readable medium, for example, may include non-volatile memory, such as Floppy, ROM, Flash memory, Disk drive memory, CD-ROM, and other permanent storage. It is useful, for example, for transporting information, such as data and computer/programming instructions, between computer systems. Furthermore, the computer readable medium may comprise computer readable information in a transitory state medium such as a network link and/or a network interface, including a wired or wireless network that allows a computer to read such computer readable information.

A novel and efficient computer-facilitated system and method for identifying one or more healthcare providers who have treated, or are likely to treat, at least one patient meeting a set of predetermined medical criteria and for identifying at least one patient meeting the set of predetermined medical criteria has been provided. Once identified, such information may be stored on a storage medium and provided to those responsible for introducing or educating healthcare providers about the healthcare therapy in order each an increasing number of healthcare providers and appropriate patients. Other features of the invention have been disclosed, but are not limited to the particular details disclosed herein.

What is claimed is:

1. A method of improving healthcare therapy recommendations, the method comprising:
establishing a health-ye dataset including at least one set of predetermined medical criteria associated with at least one identifiable healthcare therapy;
identifying by a processor a plurality of therapy healthcare providers who treated at least one patient meeting the predetermined set of medical criteria and an identifier of at least one patient meeting the set of predetermined medical criteria; and
storing at least one of the plurality of therapy healthcare providers and the identifier of the at least one patient meeting the set of predetermined medical criteria on a storage medium.

2. The method of claim 1, wherein identifying the plurality of therapy healthcare providers comprises comparing therapy criteria with a plurality of patient records in the healthcare dataset.

3. The method of claim 2, wherein comparing the therapy criteria comprises comparing a primary therapy criteria with at least one primary patient medical parameter, and comparing a secondary therapy criteria with at least one secondary patient medical parameter.

4. The method of claim 1, further comprising, subsequent to storing at least one of the plurality of therapy healthcare providers and the identifier of the at least one patient, alerting a general healthcare provider associated with the at least one patient meeting the set of predetermined medical criteria, and indicating a matching therapy and therapy provider of the therapy.

5. The method of claim 1, wherein establishing the healthcare dataset comprises receiving the healthcare dataset from a general healthcare provider, and receiving the medical criteria from a therapy developer.

6. The method of claim 1, wherein the method is performed by at least one general healthcare provider.

7. The method of claim 1, wherein identifying the plurality of therapy healthcare providers who treated at least one patient meeting the predetermined set of medical criteria comprises scoring a comparison of patent medical parameters with the set of medical criteria, and identifying patients whose patient medical parameters meet or exceed a minimum score.

8. A method of facilitating therapy recommendations to patients, comprising:
comparing, by a processor, for each of a plurality of patients, a healthcare dataset associated with the patient with at least one medical criteria associated with a respective one of at least one therapy;
identifying, by the processor, patients among the plurality of patients whose healthcare dataset sufficiently matches at least one medical criteria; and
indicating, by the processor, to a general healthcare provider associated with each of the identified patients whose healthcare dataset matches at least one medical criteria, including indicating an identity of a therapy healthcare provider associated with the at least one medical criteria.

9. The method of claim 8, comparing the healthcare dataset with the at least one medical criteria comprises comparing a plurality of patient primary medical parameters with corresponding primary values or ranges in the medical criteria for each respective one of the plurality of patient primary medical parameters.

10. The method of claim 9, further comprising, in comparing the plurality of patient primary medical parameters with the corresponding primary values or ranges for each respective one of the plurality of patient primary medical parameters, generating a score indicating a degree to which the plurality of patient primary medical parameters matches its respective corresponding primary value of the primary medical criteria.

11. The method of claim 9, further comprising comparing a plurality of patient secondary medical parameters with corresponding secondary values or ranges for each respective one of the plurality of patient primary medical parameters.

12. A system of improving healthcare therapy recommendations comprising:
at least one database storing a healthcare dataset including at least one set of predetermined medical criteria;
a processing device communicatively coupled to the at least one database;
a user-input interface communicatively coupled to the processing device, the user-input interface configured to receive input instructions from the user to identify a plurality of healthcare providers who treated at least one patient meeting the set of predetermined medical criteria and identify at least one patient meeting the set of predetermined medical criteria; and
a memory configured to store at least one of the plurality of healthcare providers and the at least one patient meeting the set of predetermined medical criteria on a storage medium.

13. The system of claim 12, wherein the processing device is configured, by execution of computer instruction code, to:
compare, for each one of at least one patient, a healthcare dataset associated with the at least one patient with the predetermined medical criteria associated with a respective one of at least one therapy; and
indicate to a general healthcare provider associated with each of the identified patients whose healthcare dataset matches at least one medical criteria of the predetermined medical criteria, including indicating an identity of a therapy healthcare provider associated with the at least one medical criteria.

14. The system of claim 13, wherein the processing device is further configured to compare a plurality of patient primary medical parameters with corresponding primary values or ranges in the medical criteria for each respective one of the plurality of patient primary medical parameters.

15. The system of claim 14, wherein the processing device is further configured to compare generate a score indicating a degree to which the plurality of patient primary medical parameters matches its respective corresponding primary value of the primary medical criteria.

16. The system of claim 13, wherein the processing device is further configured to compare a plurality of patient secondary medical parameters with corresponding secondary values or ranges for each respective one of the plurality of patient primary medical parameters.
